(19) United States
(12) Patent Application Publication
(10) Pub. No.: US 2024/0175838 A1
(43) Pub. Date: May 30, 2024

(54) HALOGENATED GAS SENSOR

(71) Applicant: INFICON GMBH, Koln (DE)

(72) Inventor: Gang GU, East Syracuse, NY (US)

(21) Appl. No.: 18/285,306

(22) PCT Filed: Mar. 17, 2022
(86) PCT No.: PCT/EP2022/057018

§ 371 Date: Oct. 2, 2023

Related U.S. Application Data
(60) Provisional application No. 63/171,661, filed on Apr. 7, 2021.

(51) Int. Cl. G01N 27/12; G01N 33/00

(52) U.S. Cl. CPC G01N 27/125; G01N 33/0027

(57) ABSTRACT

Halogenated gas sensor for detecting halogenated gas, comprising at least a first metal electrode and a second metal electrode, which are connected with a sensing material, which comprises at least one of NaAlSiO4, KAlSiO4, RbAlSiO4, CSAlSiO4.

### BACKGROUND OF THE INVENTION

Materials with at least one component with a chemical formula of NaAlSiO4 or KAlSiO4 or RbAlSiO4 or CsAlSiO4 were used to fabricate bead type sensor for highly sensitive and selective sensor for the detection of halogenated gas. The sensor contains a center electrode, a coil and sensing material. The coil is heated by current passing through it. The sensing material is porous. The conductance between center electrode and heated coil changes with concentration of halogenated gas.

This invention relates to improvement of bead type sensor for highly sensitive detection of halogenated gases, especially refrigerant gases such as hydrofluoroolefin (HFO) and hydroflurocarbons (HFCs), which show a much less global warming potential compared to hydrochlorofluorocarbons (HCFCs).

There are several existing technologies for halogenated gas detection. Tin oxide based metal oxide semiconductor (MOS) sensors have been used for halogenated gas detection; however these sensors show cross sensitivity to many hydrocarbons and humidity. Non-dispersive infrared (NDIR) optical sensors are used for halogenated gas detection as well; however these sensors show limited sensitivity and are expensive to produce.

Solid state bead type sensors to detect halogenated gases are relatively cheap to produce. These sensors are disclosed by Loh in U.S. Pat. No. 3,751,968, by Lee in U.S. Pat. No. 5,104,513, by Stetter in U.S. Pat. No. 5,226,309 and by Yannopoulos in U.S. Pat. No. 5,932,176. Loh disclosed a sensing element comprises a glass-ceramic comprising a mixture of lanthanum oxide, lanthanum fluoride, and sodium silicate. Lee disclosed a sensing element of a ceramic, comprising a mixture of potassium silicate and compound selected from the group of silicon dioxide and aluminum oxide. Stetter disclosed a sensing material comprising sodium lanthanum fluoride silicate, having the chemical formula NaLa(SiO4)3F. Yannopoulos disclosed a sensing element comprising of sodium titanate.

The operating temperature of sensor described with U.S. Pat. No. 5,226,309 is from 500° C. to 600° C., which is too low to detect HFOs and HFCs with decent sensitivity. Lee disclosed a sensing element of ceramic, comprising a mixture of potassium silicate and aluminum oxide in a ratio of between about 0.25-4.0 parts potassium silicate by weight to 1 part aluminum oxide by weight. The ratio is broad and the phase of sensing material is not well defined, hence a reproducible sensor performance is not easy to achieve. The object of this invention is to find defined materials for selective and sensitive detection of HFOs and HFCs. The other object of this invention is to find sensing materials with high melting temperature, therefore operating of the sensor with high sensitivity at high temperature from 800 C to 1000 C is possible.

The halogenated gas sensor of the invention is defined by independent claim 1. Accordingly, the gas sensor comprises at least a first metal electrode and a second metal electrode, which are connected with a sensing material, which comprises at least one of NaAlSiO4, KAlSiO4, RbAlSiO4, CSAlSiO4.

The sensing material may be in the form of a bead, in which the two electrodes are at least partially embedded.

One of said two electrodes may be a coil surrounding the other of the two electrodes as a center electrode.

The first electrode may be made of or comprises platinum and/or where the second electrode may be made of or comprises platinum.

A voltage source may be connected to at least the first electrode to heat the electrode by current or voltage applied to said electrode to a temperature in the range of 400° C.-1200° C., 400° C.-1000° ° C., 600° C.-1200° ° C. or 600° C.-1000° C.

The first sensor may be in the form of a coil having a first end and an opposing second end, said two ends being connected to a voltage source (1), said second electrode being a center electrode in the form of a longitudinal straight element or bar extending through the center of the coil (4) along the longitudinal axis of the coil, said coil and center electrode being surrounded by and embedded in said sensing material.

The invention also provides for a method of detecting halogenated gas with a sensor as described above, wherein said sensing material is heated to a temperature in the range of 400° C.-1000° C., 400°-1200° ° C., 600° C.-1000° ° C. or 600° C.-1200° C. by applying a current or voltage to said first sensor or coil.

The current through the coil after exposing the sensor to the gas to be detected may be divided by the current through the coil before the sensor is exposed to the gas to be detected.

Moreover, the invention provides for a method of manufacturing a halogenated gas sensor as described above, wherein the sensing material comprises at least a first component (A) made from a molecular sieve (3A), which is heated to a first temperature of several hundred ° C., maintained at said first temperature for several, and preferably 3 hours, thereafter being heated to a second temperature, which is higher than the first temperature and kept at said second temperature for a second time, which preferably corresponds to said first time.

Said first component may be subsequently ground to fine particles with an average size of below 5 μm, preferably about 3 μm.

Said first component may contain NaAlSiO4 and KAlSiO4, preferably at a ratio of 1:1.

The sensing material may comprise at least a second component (B) which is prepared with an ion exchange performed with a molecular sieve (4A) and CsNO₃, said molecular sieve and CsNO₃ preferably being mixed in deionized water.

The mixed suspension of molecular sieve, CsNO₃ and deionized water may be stirred for several, and preferably 24, hours, whereafter the suspension is preferably centrifuged, thereafter preferably being heated to a first temperature of several hundred ° C. and preferably about 900ºC for at least one hour and preferably for two hours, and thereafter heated to a second temperature higher than said first temperature, preferably to 1100° C., for several additional hours, and preferably for about 3 hours.

Said second component (B) may be ground after said heat treatment to fine particles with an average size of a few and preferably about 4 μm.

Said first component (A) and/or said second component (B) may be mixed with a vehicle to a slurry, said vehicle preferably being in the range of 5%-10% weight hydroxypropyl cellulose dissolved in water, the weight ratio of the mixture of said components (A) and/or (B) to said vehicle being about 2:1.

### BRIEF DESCRIPTION OF THE DRAWINGS

In the following exemplary embodiments of the invention are described with reference to the figures, in which

FIG. 1 is a schematic view of a sensor according to an embodiment of the invention,

FIG. 2 shows the steps of a typical process to fabricate the sensor with exemplic sensing materials,

FIG. 3 shows scanning electron microscopy images with 30 and 1600 magnifications,

FIG. 4 shows a typical sensor response to 100 ppm R134a and 100 ppm R1234yf,

FIG. 5 shows a typical sensor response to different concentration of R134a, and

FIG. 6 shows a typical sensor response to different gas/vapor.

### DETAILED DESCRIPTION OF THE INVENTION

### Sensor Components

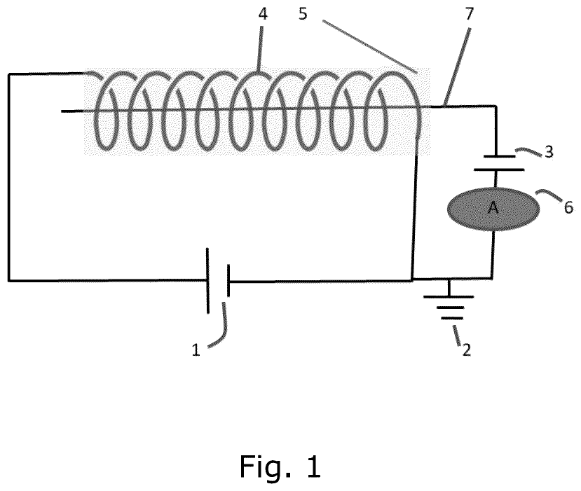

Schematic diagram of FIG. 1 shows the invented sensor comprises a center electrode 7, preferably a platinum wire, which is surrounded with a metal coil 4, preferably a platinum coil. Both are embedded in bead 5, which is comprised of at least one of NaAlSiO4, KAlSiO4, RbAlSiO4, CSAlSiO4. The coil is heated by applied voltage 1 to temperature from 400° C. to 1000° ° C. The current 6 is created with an applied voltage 3 between center electrode 7 and coil 4. The sensor nominal resistance is ratio of voltage 2 and current 6, which changes with halogenated gas concentration surrounding the sensor.

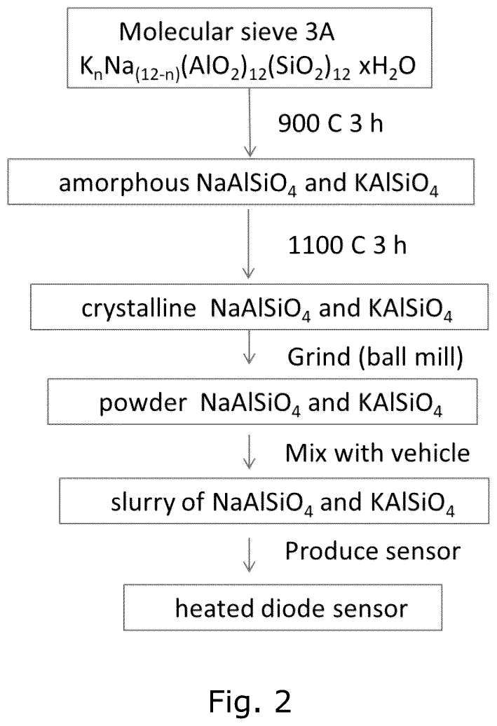

Material A for the bead 5 is synthesized with starting material of molecular sieve 3A with linear formula KnNa12-n[(AlO2)12(SiO2)12]·xH2O (n about 6) from Alfa Aesar. The process for sensor fabrication is shown in FIG. 2. For example, 50 g molecular sieve 3A was placed in a furnace and heated to 900 C with a ramping rate of 5° C./min and kept at 900° C. for 3 hours, afterwards was heated to 1100° C. with a ramping rate of 5° C./min and kept at the same temperature for 3 hours. Waiting for the material cool to room temperature, it was ground with a planetary ball mill (Retsch PM100) to fine particles with an average size about 3 um. XRD diffraction spectra of ground material were collected with PANalytical X'Pert PRO XRD system. It is confirmed that it only contains NaAlSiO4, KAlSiO4. Element analysis with energy-dispersive X-ray spectroscopy (EDS) confirmed that ratio of NaAlSiO4 to KAlSiO4 is 1:1. Material B was prepared with ion exchange, which was performed with molecular sieve 4A (Na12[(AlO2)12(SiO2)12]·nH2O from Alfa Aesar) and CsNO3. Typically 5 g molecular sieve 4A and 16.5 g CsNO3 mixed in 85 mL deionized water. The pH value was adjusted to 8 with NaOH solution. The suspension was stirred for 24 hours. Afterwards the suspension was centrifuged. The remaining material was ion exchanged with 16.5 g CsNO3 and 85 mL water solution for additional two times, the final remaining material was washed with deionized water for 3 times and dried at 80° C. in air overnight. Additional heat-treatment in air was performed to the material, which includes heating to 900° C. for 2 hours and additional 3 hours at 1100° C. After the heat-treatment, the material was ground with pestle and mortar. Fine particles with average size of about 4 um are obtained. Element analysis with energy-dispersive X-ray spectroscopy (EDS) indicated that ratio of NaAlSiO4 to CsAlSiO4 is 1:1. NaAlSiO4 and KAlSiO4 show melting points of 1526° C. and 1750° C., respectively. Therefore they are expected to show long lifetime with operating temperature of up to 1000° C.

### Sensor Fabrication

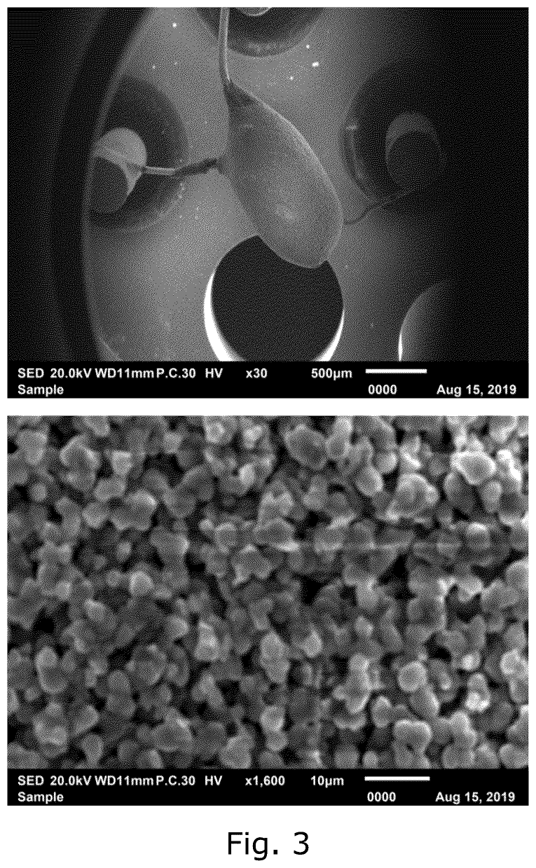

Material A or (and) Material B was (were) mixed with vehicle to get slurry. The vehicle is 5% to 10% (weight) hydroxypropyl cellulose dissolved in water. Weight ratio of Material to vehicle is about 2:1. The slurry is coated on center electrode first and wait until the coating is dry. Then the coated center electrode is inserted to heating coil. Additional slurry is added around coil to form a complete bead to cover heating coil. Afterwards the finished sensor was heated to 850° C. for 0.5-2 hours with ramping rate of 1-5° C./min. After sensors cool to room temperature, they are ready for tests. Sensor prepared with Material A/B is denoted as Sensor A/B. FIG. 3 shows SEM images of a typical Sensor A with 30 and 1600 magnification. High magnification image shows very porous morphology.

### Sensor Performance

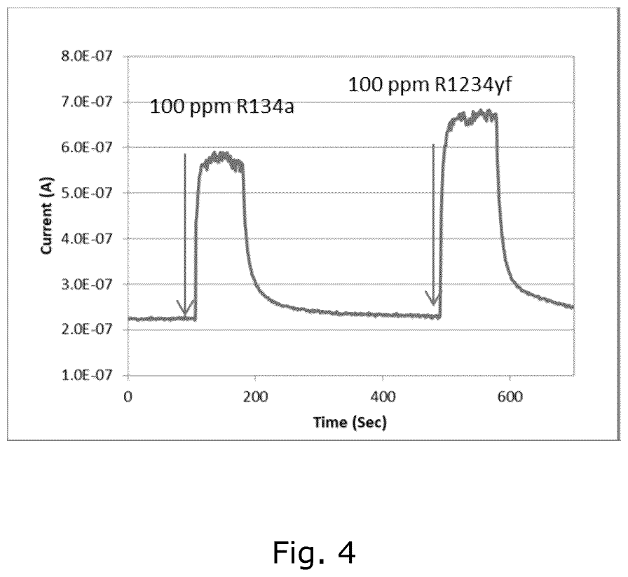

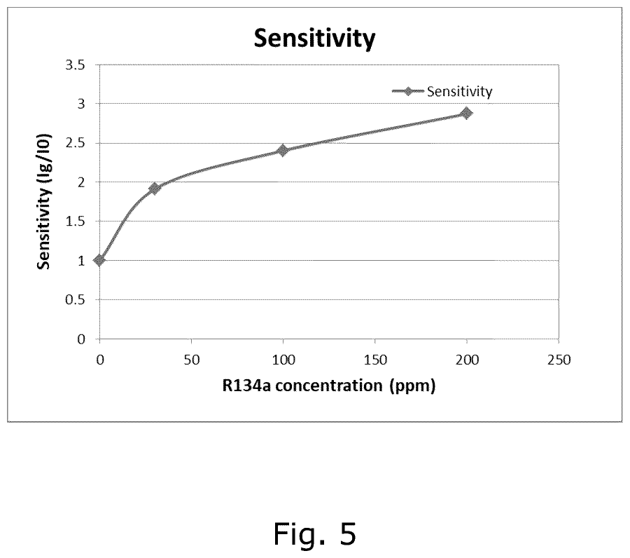

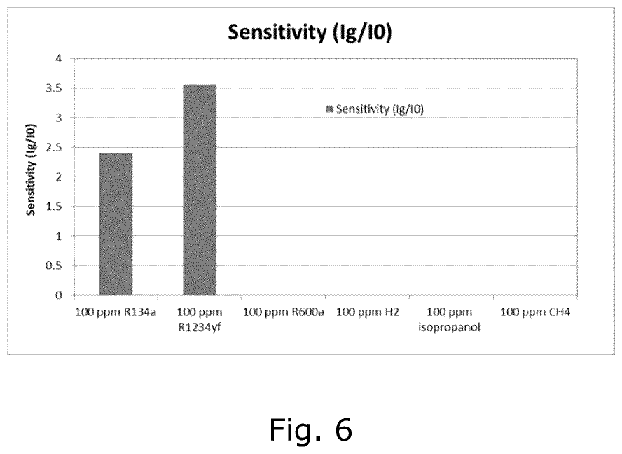

Sensor A is heated to about 800° C. by passing current through coil during operation. The current (6) in FIG. 1 changes with refrigerant gas concentration. The Current before gas exposure is denoted as I0 and the current after gas exposure is denoted as Ig. The ratio of Ig/10 is defined as sensitivity. FIG. 4 shows that Sensor A responses very quickly to 100 ppm R134a (CH2FCF3) and 100 ppm R1234yf (CF3CF═CH2). FIG. 5 shows Sensor A's sensitivity to different concentration of R134a. Leak Detector with a typical Sensor A can detect 0.6 g/year leak rate of R134a intermittently for at least 3 months. FIG. 6 shows sensitivity of Sensor A to 100 ppm different gas/vapor. The sensor shows no response to 100 ppm of isobutene (R600a), hydrogen, isopropanol and methane (CH4), but good response to 100 ppm R134a and 100 ppm R1234yf. Sensors with Material B show similar sensor performance as Sensor A, moreover it is possible to operate Sensor B at higher temperature from 800 C to 1200 C to get high sensitivity.

In the foregoing specification, the invention is described with reference to specific embodiments thereof, but those skilled in the art will recognize that the invention is not limited thereto. Different features and aspects of the above-described disclosure may be used individually or jointly.

1. A halogenated gas sensor for detecting halogenated gas, comprising at least a first metal electrode and a second metal electrode, which are connected with a sensing material, which comprises at least one of NaAlSiO4, KAlSiO4, RbAlSiO4, CSAlSiO4.

2. The sensor according to claim 1, wherein the sensing material is in the form of a bead, in which the two electrodes are at least partially embedded.

3. The sensor according to claim 1, wherein one of said two electrodes is a coil surrounding the other of the two electrodes as a center electrode.

4. The sensor according to claim 1, wherein the first electrode is made of or comprises platinum and/or where the second electrode is made of or comprises platinum.

5. The sensor according to claim 1, wherein a voltage source is connected to at least the first electrode to heat the electrode by current or voltage applied to said electrode to a temperature in the range of 400° C.-1200° C., 400° C.-1000° C., 600° C.-1200° ° C. or 600° C.-1000° C.

6. The sensor according to claim 1, wherein the first sensor is in the form of a coil having a first end and an opposing second end, said two ends being connected to a voltage source, said second electrode being a center electrode in the form of a longitudinal straight element or bar extending through the center of the coil along the longitudinal axis of the coil, said coil and center electrode being surrounded by and embedded in said sensing material.

7. A method of detecting halogenated gas with a sensor according to claim 1, wherein said sensing material is heated to a temperature in the range of 400° C.-1000° C., 400°-1200° ° C., 600° C.-1000° C. or 600° C.-1200° ° C. by applying a current or voltage to said first sensor or coil.

8. The method of detecting halogenated gas according to claim 7, wherein the current through the coil after exposing the sensor to the gas to be detected is divided by the current through the coil before the sensor is exposed to the gas to be detected.

9. A method of manufacturing a halogenated gas sensor according to claim 1, wherein the sensing material comprises at least a first component (A) made from a molecular sieve (3A), which is heated to a first temperature of several hundred ° C., maintained at said first temperature for several, and preferably 3 hours, thereafter being heated to a second temperature, which is higher than the first temperature and kept at said second temperature for a second time, which preferably corresponds to said first time.

10. The method according to claim 9, wherein said first component is subsequently ground to fine particles with an average size of below 5 μm, preferably about 3 μm.

11. The method according to claim 9, wherein said first component contains NaAlSiO4 and KAlSiO4, preferably at a ratio of 1:1.

12. The method according to claim 9, wherein the sensing material comprises at least a second component (B) which is prepared with an ion exchange performed with a molecular sieve and CsNO3, said molecular sieve and CsNO3 preferably being mixed in deionized water.

13. The method according to claim 12, wherein the mixed suspension of molecular sieve, CsNO3 and deionized water is stirred for several, and preferably 24, hours, whereafter the suspension is preferably centrifuged, thereafter preferably being heated to a first temperature of several hundred ° C. and preferably about 900° C. for at least one hour and preferably for two hours, and thereafter heated to a second temperature higher than said first temperature, preferably to 1100° C., for several additional hours, and preferably for about 3 hours.

14. The method according to claim 13, wherein said second component (B) is ground after said heat treatment to fine particles with an average size of a few and preferably about 4 μm.

15. The method according to claim 9, wherein said first component (A) and/or said second component (B) are mixed with a vehicle to a slurry, said vehicle preferably being in the range of 5%-10% weight hydroxypropyl cellulose dissolved in water, the weight ratio of the mixture of said components (A) and/or (B) to said vehicle being about 2:1.
